(19) United States
(12) Patent Application Publication
(10) Pub. No.: US 2006/0177839 A1
(43) Pub. Date: Aug. 10, 2006

(54) Method for modulating the evolution of a polypeptide encoded by a nucleic acid sequence

(76) Inventors: Didier Mazel, Suresnes (FR); Guillaume Cambray, Paris (FR)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 11/228,291

(22) Filed: Sep. 19, 2005

Related U.S. Application Data
(60) Provisional application No. 60/610,597, filed on Sep. 17, 2004.

(51) Int. Cl. C12Q 1/68; G06F 19/00

(52) U.S. Cl. 435/6; 702/20

(57) ABSTRACT

A method for modulating the ability of a gene to mutate by analyzing codon usage within the gene and selecting a synonymous nucleotide sequence with a higher, lower or different capacity to mutate. The method permits widening and optimization of the evolutionary landscape of a protein. A computer-implemented method for analyzing and selecting nucleotide sequences with an altered ability to mutate.

### CROSS-REFERENCE TO RELATED APPLICATIONS

This application claims priority under 35 U.S.C. §119(e) to U.S. Provisional Application 60/610,597, filed Sep. 17, 2004 and to U.S. Provisional Application attorney docket number 278662USOPROV, filed Sep. 19, 2005.

### REFERENCE TO MATERIAL ON COMPACT DISK

An example of the ELP program and ELP program out is provided on the compact disk attached to this application. The contents of this compact disk form part of this disclosure and are also incorporated by reference.

### BACKGROUND OF THE INVENTION

1. Field of the Invention

A method for modulating the ability of a gene to mutate by analyzing codon usage within the gene and selecting a synonymous nucleotide sequence with a higher, lower or different capacity to mutate. A computer-implemented method for analyzing and selecting nucleotide sequences with an altered ability to mutate. Mutate is here defined at the level of amino-acid sequence. Mutation then does not refer to nucleotide as usual but to amino-acid changes. Consequently silent or neutral mutations of a codon must not to be considered.

2. Description of the Related Art

The genetic code is known. This code is redundant. That is, for most polypeptides, there are many different nucleic acid sequences that encode the same amino acid sequence forming a polypeptide or protein.

The table below shows the genetic code and which codons encode which amino acids. The codons UAA, UGA and UAG are stop codons in the standard genetic code and do not ordinarily encode an amino acid. The table below shows each codon and the amino acid it encodes. For example: UUU encodes phenylalanine (Phe, F) and UCU encodes serine (Ser, S).

| | | |
|---|---|---|
| First | Second Position of Codon | Third |

| | | | | | |
|---|---|---|---|---|---|
| Position | U | C | A | G | Position |

| | | | | | |
|---|---|---|---|---|---|
| U | UUU | UCU | UAU | UGU | U |
| | Phe | Ser | Tyr | Cys | C |
| | [F] | [S] | [Y] | [C] | A |
| | UUC | UCC | UAC | UGC | G |
| | Phe | Ser | Tyr | Cys | |
| | [F] | [S] | [Y] | [C] | |
| | UUA | UCA | UAA | UGA | |
| | Leu | Ser | Ter | Ter | |
| | [L] | [S] | [end] | [W] | |
| | UUG | UCG | UAG | UGG | |
| | Leu | Ser | Ter | Trp | |
| | [L] | [S] | [end] | [end] | |
| C | CUU | CCU | CAU | CGU | U |
| | Leu | Pro | His | Arg | C |
| | [L] | [P] | [H] | [R] | A |
| | CUC | CCC | CAC | CGC | G |
| | Leu | Pro | His | Arg | |
| | [L] | [P] | [H] | [R] | |
| | CUA | CCA | CAA | CGA | |
| | Leu | Pro | Gln | Arg | |
| | [L] | [P] | [Q] | [R] | |
| | CUG | CCG | CAG | CGG | |
| | Leu | Pro | Gln | Arg | |
| | [L] | [P] | [Q] | [R] | |
| A | AUU | ACU | AAU | AGU | U |
| | Ile | Thr | Asn | Ser | C |
| | [I] | [T] | [N] | [S] | A |
| | AUC | ACC | AAC | AGC | G |
| | Ile | Thr | Asn | Ser | |
| | [I] | [T] | [N] | [S] | |
| | AUA | ACA | AAA | AGA | |
| | Ile | Thr | Lys | Arg | |
| | [I] | [T] | [K] | [R] | |
| | AUG | ACG | AAG | AGG | |
| | Met | Thr | Lys | Arg | |
| | [M] | [T] | [K] | [R] | |
| G | GUU | GCU | GAU | GGU | U |
| | Val | Ala | Asp | Gly | C |
| | [V] | [A] | [D] | [G] | A |
| | GUC | GCC | GAC | GGC | G |
| | Val | Ala | Asp | Gly | |
| | [V] | [A] | [D] | [G] | |
| | GUA | GCA | GAA | GGA | |
| | Val | Ala | Glu | Gly | |
| | [V] | [A] | [E] | [G] | |
| | GUG | GCG | GAG | GGG | |
| | Val | Ala | Glu | Gly | |
| | [V] | [A] | [E] | [G] | |

As shown above, different codons may encode the same amino acid. For example, in the standard genetic code there are six codons which encode leucine (Leu, L). These codons are known as synonymous codons, because they each encode the same amino acid. While synonymous codons encode the same amino acid residue, each organism has a preference for particular synonymous codons over others. This preference is known as codon bias. For example, according to Source: www.tigr.ory Escherichia coli, strain K-12 exhibits the following codon usage:
Escherichia coli K12 [gbbct]: 5095 CDS's (1609357 codons)

[AA] [codon] [Triplet Frequency for corresponding AA]

| | | |
|---|---|---|
| Ala | GCA | 21.32% |
| Ala | GGT | 16.14% |
| Ala | GCG | 35.56% |
| Ala | GCC | 26.98% |
| Arg | CGG | 9.85% |
| Arg | CGA | 6.47% |
| Arg | AGA | 3.85% |
| Arg | CGT | 37.78% |
| Arg | AGG | 2.25% |
| Arg | CGC | 39.80% |
| Asn | AAC | 54.88% |
| Asn | AAT | 45.12% |
| Asp | GAT | 62.78% |
| Asp | GAC | 37.22% |
| Cys | TGT | 44.43% |
| Cys | TGC | 55.57% |
| End | TAA | 63.08% |
| End | TAG | 7.61% |
| End | TGA | 29.31% |
| Gln | CAA | 34.77% |
| Gln | CAG | 65.23% |
| Glu | GAG | 31.14% |
| Glu | GAA | 68.86% |
| Gly | GGG | 15.11% |
| Gly | GGA | 10.90% |
| Gly | GGC | 40.33% |
| Gly | GGT | 33.66% |
| His | CAT | 57.11% |
| His | CAC | 42.89% |
| Ile | ATA | 7.33% |
| Ile | ATT | 50.71% |
| Ile | ATC | 41.96% |
| Leu | CTG | 49.52% |
| Leu | TTG | 12.88% |
| Leu | CTC | 10.44% |
| Leu | CTA | 3.68% |
| Leu | TTA | 13.10% |
| Leu | CTT | 10.38% |
| Lys | AAA | 76.51% |
| Lys | AAG | 23.49% |
| Met | ATG | 100.00% |
| Phe | TTC | 42.58% |
| Phe | TTT | 57.42% |
| Pro | CCG | 52.50% |
| Pro | CCC | 12.47% |
| Pro | CCA | 19.11% |
| Pro | CCT | 15.92% |
| Ser | TCA | 12.38% |
| Ser | TCC | 14.84% |
| Ser | AGT | 15.15% |
| Ser | TCT | 14.55% |
| Ser | TCG | 15.40% |
| Ser | AGC | 27.67% |
| Thr | ACC | 43.39% |
| Thr | ACA | 13.19% |
| Thr | ACT | 16.64% |
| Thr | ACG | 26.78% |
| Trp | TGG | 100.00% |
| Tyr | TAT | 56.99% |
| Tyr | TAC | 43.01% |
| Val | GTC | 21.54% |
| Val | GTG | 37.28% |
| Val | GTT | 25.80% |
| Val | GTA | 15.38% |

In the same manner codon (triplet) frequency for corresponding amino acids for humans or other organisms can be easily obtained from their correspondent codon bias.

A native gene will generally tend to exhibit the codon usage or preference of the particular organism from which it is derived. However, the codons of a native or original gene sequence are limited to the sequence space that they can explore and then to the amino acid they can reach. Thus, said original codons are not necessarily the codons with the highest or broadest capacity to mutate.

By “sequence space” of a defined nucleotide sequence, we intend all possible nucleotide sequences derived by a single point mutation of one single codon of the original sequence.

As disclosed below, however, not all codons encoding the same amino acid residue are equivalent. Some synonymous codons allow for a greater frequency or range of mutation than others. The present invention is based in part on replacing the codons in a native protein-coding sequence with synonymous codons with a higher, broader or different capacity to mutate.

Codon usage and bias has been studied for frequency-dependent selection of epitopes in pathogens such as influenza virus, Plotkin et al., Proc Natl Acad Sci U S A. 2003 Jun. 10; 100(12):7152-7. Epub 2003 May 14. Codon volatility has been used to measure selective pressures on proteins, Plotkin et al. Nature vol 428 29 April 2004. Codon usage and bias have been used to passively analyze known gene sequences or construct phylogenetic trees, in order to analyze past history of the sequence. However, methods of using such information to engineer new nucleotide sequences having a modified capacity to mutate have not previously been suggested. In other words, manipulation of a given gene's codon usage has never been proposed to alter its subsequent evolution.

The present invention is based on the discovery that by replacing one or more codons in a native or original polypeptide-encoding nucleic acid sequence (gene) by a synonymous codon, the subsequent evolution of the polypeptide-encoding nucleic acid sequence can be controlled. Indeed some amino acids that were unreachable by way of a single point mutation can be reached from an alternative synonymous sequence. Hence, the method renders certain mutations evolutionary accessible. Some protein mutants, which were virtually unobtainable (evolutionarily inaccessible) using the wild-type or original nucleic acid sequence, become possible when an appropriate synonymous nucleic acid sequence is used.

The method of the present invention can be used to increase, decrease, stabilize or change the ability of a native gene to mutate. Increasing the mutational frequency or altering the range of mutations that can occur in a polypeptide-encoding nucleic acid sequence is beneficial when further selecting for functional variants of the protein encoded by the original or native nucleic sequence.

The method may also be used to reduce the mutational frequency of a nucleic acid sequence or gene, when a high mutation rate is undesirable, such as when a sequence is used to encode biologically useful proteins or vaccines.

### BRIEF SUMMARY OF THE INVENTION

One aspect of the invention is a method for controlling the mutational behavior of a nucleic acid sequence encoding a particular polypeptide based on the differences among or between the mutational capacities of synonymous codons.

Another aspect of the invention is directed to a method for selecting a synonymous nucleic acid sequence which encodes the same polypeptide as an original (e.g., native, wild-type) gene or nucleic acid sequence, but which has an altered capacity to mutate. Selection may be based on increasing, diversifying, or decreasing the mutation rate of the synonymous gene sequence. As explained below, this method may be used to select a synonymous nucleic acid sequence exhibiting the maximal relative evolutionary power or, alternatively, a sequence having the maximal intrinsic evolutionary power.

A sequence may also be selected based on its ability to undergo particular mutations, such as increasing or decreasing the mutation rate of one or more codons to mutant codons encoding a particular amino acid.

A third aspect of the invention is computer-implemented method for analyzing or determining synonymous nucleic acid sequences of a given original gene sequence that have a modified capacity to mutate. This aspect also includes computer programs or software suitable for determining or selecting the desired synonymous nucleic acid sequence, as well as a computer system which executes or implements the software or computer program. One example of computer software suitable for this purpose is the ELP software as described for example in FIG. 2.

Other aspects of the invention will be apparent from the following disclosure.

### BRIEF DESCRIPTION OF THE DRAWINGS

The patent or application file contains at least one drawing executed in color. Copies of this patent or patent application publication with color drawing(s) will be provided by the office upon request and payment of the necessary fee.

FIG. 1 shows the evolutive (evolutionary) landscape for the UUG and CUC codons.

FIG. 2 shows an ELP (Evolutionary Landscape Painter) working diagram.

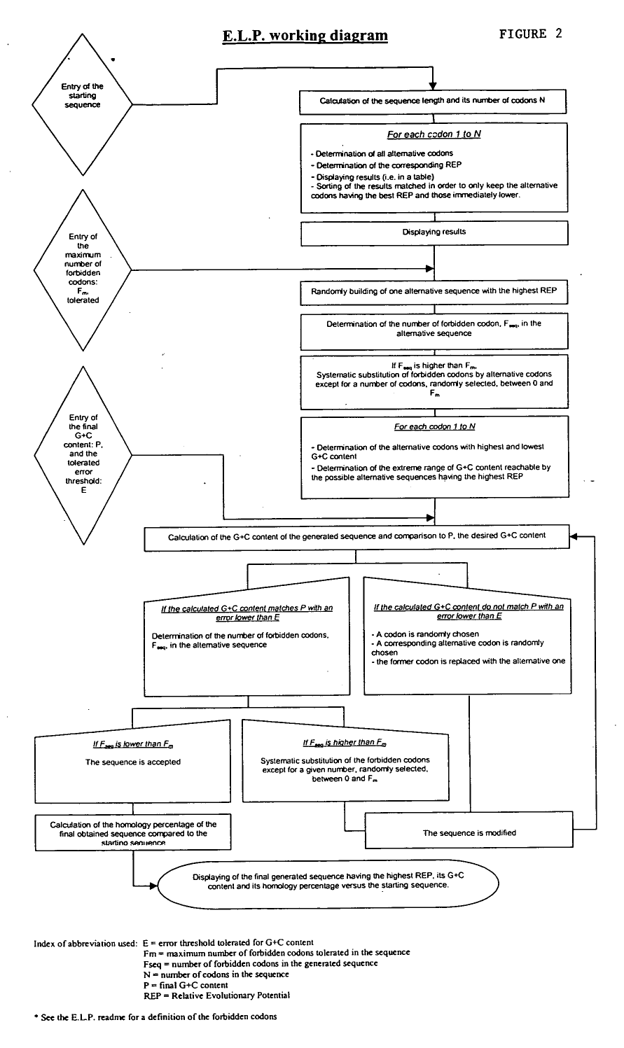

FIG. 3 depicts the dfrBI wild type (low GC content) and dfrB1_{GC} (high GC content) nucleic acid sequences. Both nucleic acid sequences encode the same amino acid sequence (blue). Modifications to the original dfrB1 nucleotide sequence are shown in red.

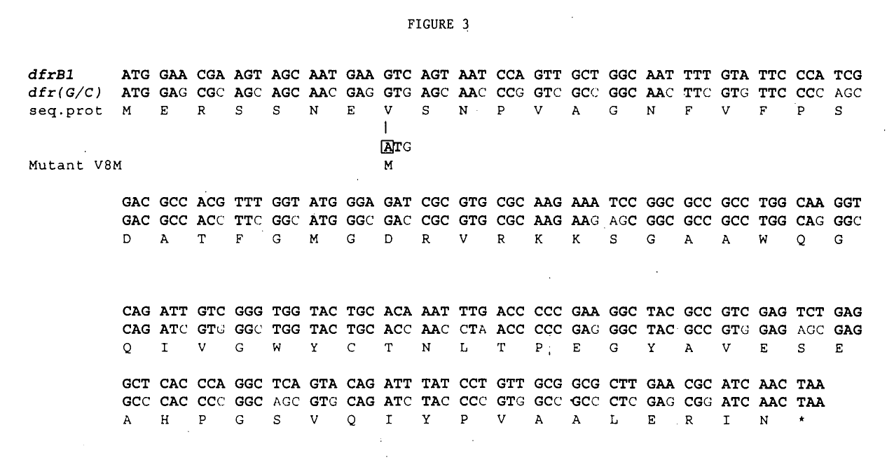

FIG. 4 illustrates a computer system 1201 upon which an embodiment of the present invention may be implemented.

FIG. 5 (color) depicts an evolutionary landscape. Original amino acid residues are shown in pink. Residues accessible by mutation of the original (red), synthetic (blue), both original and synthetic (yellow) or not accessible by a single mutation event (white) are shown.

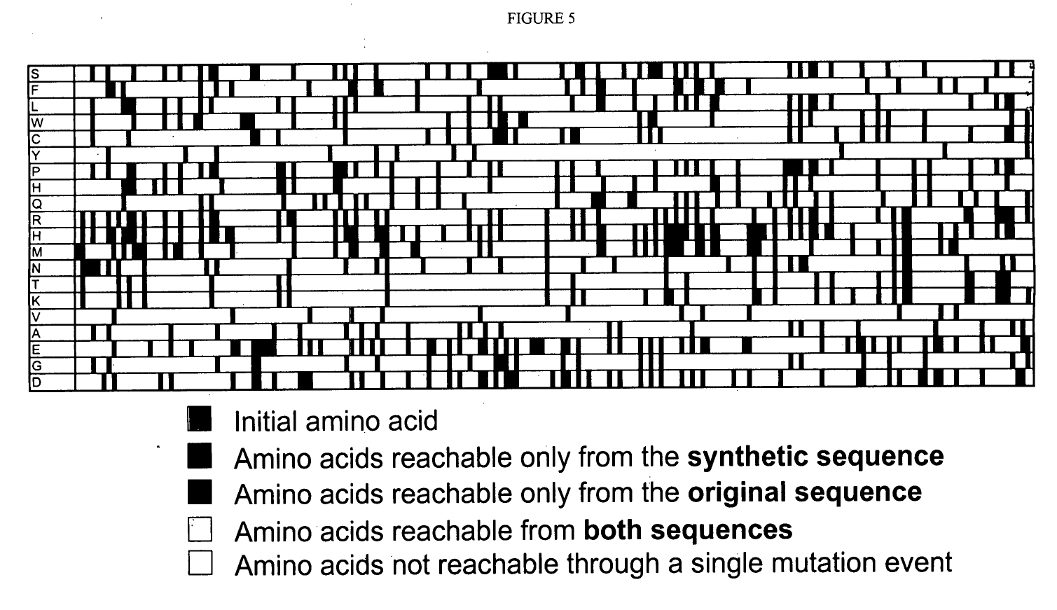

### DETAILED DESCRIPTION OF THE INVENTION

An original nucleic acid sequence may be isolated and sequenced based on methods well-known in the art as described, for example, by Current Protocols in Molecular Biology, (April, 2004, through supplement 66), see e.g., Chapter 2 “Preparation and Analysis of DNA” and Chapter 7 “DNA Sequencing”. Alternatively, the nucleotide sequence for a particular gene and the actual or deduced amino acid sequence encoded by that gene may have already been published or be available from a sequence database. Numerous nucleotide sequences of both prokaryotic and eukaryotic organisms are known. For example, GenBank® is the NIH genetic sequence database, an annotated collection of all publicly available DNA sequences (Nucleic Acids Research 2004 Jan 1 ;32(1):23-6). There are approximately 37,893,844,733 bases in 32,549,400 sequence records as of February 2004. This database is hereby incorporated by reference. Other sequence databases are incorporated by reference to Current Protocols in Molecular Biology (April, 2004, through supplement 66), Chapter 19 “Informatics for Molecular Biologists”.

Once a nucleotide sequence of interest has been identified, if the corresponding amino acid sequence is not already known, it may be easily deduced based on the structure of the nucleotide sequence referring to the genetic code. Computer programs suitable for this purpose are well-known and are incorporated by reference to Current Protocols in Molecular Biology (April, 2004, through supplement 66), Chapter 19 “Informatics for Molecular Biologists”. Alternatively the ELP program can be used.

As discussed above, an original nucleotide sequence will show a particular codon usage and codon bias generally corresponding to the organism from which it was derived. The original or wild-type nucleotide sequence does not necessarily have a high capacity to accumulate point mutations which change the identity of the amino acid sequence it encodes. However, the evolutionary ability of this native sequence may be optimized by the method of the present invention.

There are numerous synonymous nucleotide sequences encoding most polypeptides and proteins. Each particular synonymous nucleotide sequence has a particular capacity to accumulate point mutations in its codons. The present inventors have discovered a method for identifying and selecting the synonymous nucleotide sequences with a higher, lower, or simply different, capacity to mutate. For example, point mutations sustained by these engineered synonymous polynucleotide sequences provide a wider range of polypeptide mutants than would the unmodified native sequence.

Each synonymous nucleotide sequence has a potential mutation frequency based on the identity of the specific codon used to encode amino-acid at each codon position. Point mutations may be made to some synonymous codons without affecting the amino acid encoded by that codon. For example, a point mutation of the third nucleotide of the CUU leucine codon will have not affect the amino acid encoded by the mutant because CUU, CUC, CUG and CUA all encode leucine. On the other hand, other point mutations, such as to nucleotides 1 and 2 of the CUU leucine codon will cause the mutant codon to encode a different amino acid than leucine. Depending on the identity of the particular leucine codon, single point mutations will allow the resulting mutant codon to encode a range of different amino acids.

The evolutionary landscape (evolutive landscape, EL) of a particular codon refers to all the different amino acids accessible by a single point mutation of the original codon. Since different synonymous codons may have different evolutionary landscapes, each codon has a particular mutational capacity and frequency. For example, a single base mutation of leucine codon UUG could alter this codon to a codon for Phe (UUU, UUC), Leu (UUA, CUG), Met (AUG), Val (GUG), Ser (UCG), or Trp (UGG). The evolutionary landscape of the UUG codon would encompass Phe, Leu, Met, Val, Ser and Trp. Similarly, the evolutionary landscape of the adjacent UUA (Leu codon) would encompass Phe, Leu, Ile, Val, and Ser. The stop codons (UAA, UGA and UAG) are not considered as part of the evolutionary landscape because they rather stand as an evolutionary dead end.

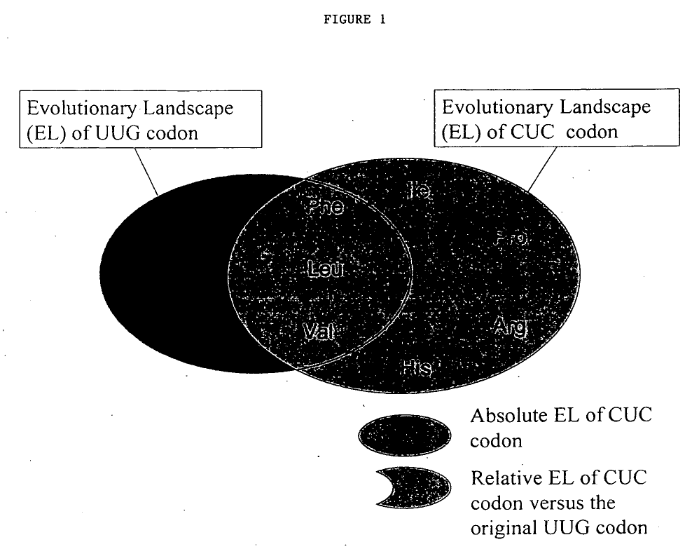

The “intrinsic evolutionary power” (IEP) of a codon is defined as the whole number of amino acids present in the evolutionary landscape of the considered codon, that is, it is equal to the cardinal number of this set of accessible amino acids. For the UUG codon the AEL is 6 (Phe, Leu, Val, Met, Ser and Trp). For the CUC codon the AEL is 7 (Phe, Leu, Val, His, Arg, Pro, Ile)—see FIG. 1 The intrinsic evolutionary power of the UUG (Leu) codon described above is six (6), because a single base mutation in this codon would allow the mutated codon to encode any one of six different amino acids. The intrinsic evolutionary power of the adjacent UUA (Leu) codon is five (5).

The “relative evolutionary power” (REP) of a codon is defined as the number of amino acids that are part of the evolutionary landscape of the alternative codon but do not form part of the evolutionary landscape of the original codon, that is, it is equal to the cardinal number EEP minus the cardinal number of the intersection between the evolutionary landscapes of the original codon and the considered codon. This intersection represents the amino acids which are part of the landscapes of both the original codon and the considered codon, in FIG. 1 these amino acids are Phe, Leu and Val.

The REP of the CUC codon would thus be +4, because a single point mutation of the CUC codon could cause it to encode four amino acids (Ile, Pro, Arg, His) not encodable by a single point mutation of the UUC codon.

The evolutionary landscape (EL) of a codon is the number of different amino acids that said codon could encode if it sustained a point mutation to a single base. For example, the evolutionary landscapes of the original codon UUG and alternates codons UUA, CUU, CUC, CUA and CUG encoding Leu are shown below.

| | | | | | | | | | | | |
|---|---|---|---|---|---|---|---|---|---|---|---|
| Codon | AA | AA | AA | AA | AA | AA | AA | AA | AA | AA | AA |

| | | | | | | | | | | | |
|---|---|---|---|---|---|---|---|---|---|---|---|
| UUA | Leu | Ser | Ile | | | | | | | Val | Phe |
| UUG | Leu | Ser | | Trp | | Met | | | | Val | Phe |
| CUU | Leu | | Ile | | | | Pro | His | Arg | Val | Phe |
| CUC | Leu | | Ile | | | | Pro | His | Arg | Val | Phe |
| CUA | Leu | | Ile | | Glu | | Pro | | Arg | Val | |
| CUG | Leu | | | | Glu | Met | Pro | | Arg | Val | |

The intrinsic evolutionary power (IEP) is the number of amino acids within the evolutionary landscape of a codon, e.g., for UAA there are five amino acids within the evolutionary landscape shown in the table above (Leu, Ser, Ile, Val and Phe).

The relative evolutionary power (REP) is the number of amino acids in the evolutionary landscape of a substitute codon that are not part of the evolutionary landscape of the original codon. If the codon in the original polynucleotide sequence is UUG, then the relative evolutionary power of the other five leucine codons compared to UUG is:

UUG

| | | |
|---|---|---|
| (Native codon) | REP | IEP |

| | | |
|---|---|---|
| UUA | +1 | 5 |
| UUG | 0 | 6 |
| CUU | +4 | 7 |
| CUC | +4 | 7 |
| CUA | +4 | 6 |
| CUG | +3 | 6 |

The algorithm developed by the inventors allows selection of the codons having the highest relative evolutionary power. The proposed method allows the selection of mutant codons that would need at least two mutations to be selected naturally. It thus modify the evolutionary landscape at a given codon position encoding a particular amino acid. Indeed, for an original UUA codon to mutate to a Met codon (AUG) it must undergo two mutations, i.e., UUA to AUA or from UUA to UUG, and then AUA to AUG or from UUG to AUG. However, by replacing the original UUA codon with the UUG codon, only a single mutation would be required to produce the AUG (Met) codon. Since double point mutations in a single codon are infrequent during mutagenesis, the present method facilitates mutation of such a sequence.

The relative evolutionary power (REP) parameter allows one to easily substitute an original codon by a synonymous codon in order to maximize the ability to explore the evolutionary landscape for that codon position. For example, if the native codon is UUG (leucine), one might replace this native codon with either UUA or CUU which are both synonymous codons for leucine. However, selection of CUU would maximize the evolutionary landscape available because CUU has a REP of +4 while UUA only has a REP of +1. That is selection of CUU would allow the possibility of point mutations to codons encoding four amino acids inaccessible by point mutations of the original UUG codon, while selection of UUA would only allows reaching one amino acid inaccessible by point mutation of the original UUG codon. The introduction of the “relative evolutionary power” parameter allows a designer to determine an alternative codon that change as most as possible the evolutionary landscape explorable at a given codon position.

A process, by means of PERL based software, can calculate values of the “relative evolutionary power” parameter for each alternative codon and then replace each original codon by one alternative codon, in order to obtain two alternative sequences based either on having maximal intrinsic evolutionary power or having maximal relative evolutionary power.

The “evolutionary powers” described so far can be considered as quantitative ones because they rely on the mere counting of reachable amino-acids. However, “qualitative evolutionary power” may also be envisaged. For instance, a specific evolutionary power can be attributed to each synonymous codon according to the needs of the designer. This way a synonymous codon may also be selected based on its absolute ability to mutate to a codon encoding any amino acid different from that of the original codon.

Alternatively a synonymous codon may be selected on the basis of its specific ability to mutate to a codon encoding one of a specific class of amino acids, such as positively-charged (basic: lysine, arginine, histidine), negatively-charged (acidic: aspartate, glutamate), non-polar (hydrophobic: glycine, alanine, valine, leucine, isoleucine, methionine, phenylalanine, tryptophan, proline) or nonionizable polar (serine, threonine, asparagine, glutamine, cysteine, selenocysteine, tyrosine). Then, a designer can define a specific table of qualitative evolutionary power that would depend on the nature of native codons in order to force selection of alternative codon of same or different nature as the native one. For example, one can decide to attribute higher evolutionary power to alternative codon leading to basic amino-acid if the native codon encodes itself a basic amino acid. In such a case, if the native codon were CGA (Arg, basic) then more power would be attributed to CGC because CAC (which encodes His, another basic amino-acid) is reachable from CGC.

Also, one can decide to attribute a less evolutionary power to some codons leading to a limited usage of particular codons, to avoid for example the use of codons that are rarely used by the host or to avoid sequences having two consecutive or contiguous “rare” codons.

Selection of a synonymous codon may also be based on its ability to mutate into a codon encoding a specific amino acid, such as to a codon encoding an amino acid with an ability to form crosslinks (cysteine), ability to form kinks (proline) in a protein, or by its capacity for post-translational modification. For example, a double point mutation of a UCU or UCG serine codon in a wild-type nucleic acid sequence would be required to convert the Ser codon to a Cys codon. However, only a single point mutation would be required to make this change in a synonymous nucleotide sequence which uses a UCU or UCC Ser codon.

Alternatively, a synonymous nucleotide sequence may be selected to reduce its capacity or frequency of mutation by selecting one or more codons with a reduced capacity to change to another amino acid or by reducing the range of amino acids encoded by a mutant codon resulting from a single base mutation of the original codon. Such a method would be advantageous for stabilizing nucleic acid sequences used to produce biologically active polypeptides or vaccines.

The relative or intrinsic evolutionary power of an original sequence may be increased (or decreased) by modifying a number of codons ranging from one codon up to all the codons of the sequence. The percentage of codons modified may be expressed as either the number of modified codons divided by the total number of codons in the original sequence, or the number of modified codons divided by the number of codons having synonymous codons within the original sequence. For example, at least 0.01, 0.1, 0.25, 0.5, 1, 2, 5, 10, 20, 30, 40, 50, 60, 70, 80, 90, 95, 99 or even 100% of the codons of a given sequence may be modified. This range includes all intermediate values and subranges and the percentage values take into account the number of codons in the original polynucleotide, e.g., the minimal percent modification for a polynucleotide having only 100 codons (300 nucleotides) would be 1%. For example, the minimal modification to be made to a polynucleotide sequence would be the replacement of a single codon, where the substituted codon has a higher or lower intrinsic or relative evolutionary power than the codon in the corresponding wild-type or native polynucleotide sequence. The maximal number of codons of a polynucleotide which may be modified would be all the codons having at least one synonymous codon encoding the same amino acid. The range of modification contemplated by the present invention is from a single codon to all the synonymous codons or any intermediate percentage of modifiable codons, where the minimal percentage is expressed as 1 over the total number of codons in the polynucleotide sequence or 1 over the total number of modifiable codons (codons having at least one synonymous codon).

Selection of a synonymous nucleotide sequence can be performed using the computer-implemented method of the invention. This method analyzes or determines synonymous nucleic acid sequences of a given original gene sequence which have a modified capacity to mutate. This aspect also includes computer programs or software suitable for determining or selecting the desired synonymous nucleic acid sequence, as well as a computer system which executes or implements the software or computer program. One example of computer software suitable for this purpose is the ELP software (ELP for Evolutionary Landscape Painter), a PERL based Software developed by the inventors. A brief description of the steps included in the ELP software is described below.

The invention is not limited to the standard genetic code, but may also be applied to genes encoded by non-standard genetic codes, such as those found in vertebrate, invertebrate, yeast, or protist mitochondria, or in the nuclear nucleic acids of certain bacteria, yeasts and ciliates. It may also be applied to nucleic acids conforming to an artificial genetic code. For example, it may be used in conjunction with the use of a nonsense mutation suppression method, which incorporates non-standard amino acids into a polypeptide.

Once a synonymous nucleotide sequence has been identified, it may be synthesized by methods well-known in the art, such as by chemical or biochemical synthesis. Methods for synthesizing nucleotide sequences are described by Current Protocols in Molecular Biology (April, 2004, through supplement 66), which is hereby incorporated by reference. For example, once the alternative sequence of the first mutated gene is obtained, the designed synthetic nucleic acid is prepared by synthesis of fragments of about 70 bp. Said fragments are 5′ end phosphorylated, consecutive, correspond to the two strands of the gene and overlap the junctions of the complementary strand. These fragments are ligated to form the longer sequence desired.

When the synonymous nucleic acid sequence has been obtained, it may be subjected to mutation. Generally, the selected synonymous nucleic acid sequence will have a higher, greater or different capacity to mutate than the original nucleic acid sequence. The selected synonymous sequence is subjected to mutagenesis, mutant sequences (which encode amino acid sequences different than the original gene) are obtained, expressed and selected or screened on the basis of a factor of interest, often a biological property such as enzymatic activity or form immunogenic or antigenic activity.

Methods for inducing point mutations in a nucleotide sequence are well-known in the art. These methods include chemical or random mutagenesis using the polymerase chain reaction (PCR), directed mutagenesis using PCR, oligonucleotide-directed mutagenesis, mutagenesis with degenerate oligonucleotides, and linker-scanning mutagenesis. One method particularly indicated for inducing hypermutation of a synonymous nucleotide sequence is by taq “error-prone” mediated hypermutation. Mutagenesis methods are also incorporated by reference to Current Protocols in Molecular Biology, Chapter 8 “Mutagenesis of Cloned DNA” (April, 2004, supplement 66).

Methods, vectors and host cells for expressing nucleic acid sequences are well-known and the methods described by Current Protocols in Molecular Biology, (April, 2004, supplement 66), which is hereby incorporated by reference, see e.g., Chapters 1-3, 5 and 6. For example, a nucleic acid sequence may be expressed by inserting it into a vector, transforming the vector into a prokaryotic or eukaryotic host cell under conditions suitable for protein expression. For example, the synthetic synonymous nucleic acid may be cloned into a low copy number vector such as ori VpSC101 and then expressed in a bacterium such as Escherichia coli.

Alternatively, the mutated nucleotide sequence may be expressed using various cell-free protocols which are known in the art. Methods for screening polypeptides encoded by mutated synonymous nucleic acid sequences involve selection on the basis of a genetic or phenotypic characteristic of the mutated polypeptide. For example, selection may be based on the biological activity of the mutant polypeptide, such as its enzymatic activity, substrate-binding activity, or immunological activity. A mutant enzyme may be tested for its absolute or relative enzymatic activity, and a mutated immunogen or antigen for its absolute or relative immunogencity or antigenicity. Mutant proteins may also be screened on the basis of their structural characteristics, such as there abilities to form certain structures like di-sulfide crosslinks or other secondary, tertiary and quaternary structures.

Natural selection may also be employed based on the ability of a cell transformed with the mutant protein to survive under particular culture conditions (for example presence of particular chemicals or antibiotics) specifically designed to positively link features of interest to cell fitness. This selection could be made by spreading out the bacteria in a selective medium or by competition in liquid cultures containing antibiotic concentrations near the limit of resistance. The phenotype and nucleotide sequence of selected mutant can be confirmed and biochemical properties of the encoded proteins further evaluated.

Methods for analyzing the biological activity and structural characteristics are well-known in the art. Many screening methods are known to those of skill in the art. Specific reference is made to such methods as disclosed by Current Protocols in Molecular Biology (April, 2004, through supplement 66), which is hereby incorporated by reference.

Once a mutant nucleic acid encoding a polypeptide mutant of interest is identified, the mutant nucleic acid sequence may be further modified by iterations of the above method. Once identified mutation of interest can also be put together on a sequence either synthesized or obtained by DNA shuffling in order to evaluate their interactions.

Mutant polypeptide sequences encoded by mutant or modified polynucleotides produced by the method of the present invention will generally have at least 90, 95 or 99% sequence similarity with the original polypeptide and will generally be encoded by polynucleotides which are at least 90, 95 or 99% similar to the polynucleotide sequence encoding the original polypeptide or a polynucleotide which is synonymous with that encoding the original polypeptide. Such mutant polypeptides may also be encoded by polynucleotide sequences which hybridize under stringent conditions to the original polynucleotide sequence or to a polynucleotide sequence synonymous with that of the original polynucleotide sequence determined by the methods of the present invention.

Such similarity may be determined by an algorithm, such as those described by Current Protocols in Molecular Biology, vol. 4, chapter 19 (1987-2004) or by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman, Advances in Applied Mathematics 2: 482-489 (1981), to find the best segment of identity or similarity between two sequences. Gap. performs global alignments: all of one sequence with all of another similar sequence using the method of Needleman and Wunsch, J. Mol. Biol. 48:443-453 (1970). When using a sequence alignment program such as BestFit, to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Such variants may also be characterized in that a nucleic acid sequence encoding such a variant will hybridize under stringent conditions with the original or synonymous polynucleotide sequence. Such hybridization conditions may comprise hybridization at 5× SSC at a temperature of about 50 to 68° C. Washing may be performed using 2× SSC and optionally followed by washing using 0.5×SSC. For even higher stringency, the hybridization temperature may be raised to 68° C. or washing may be performed in a solution of 0.1× SSC. Other conventional hybridization procedures and conditions may also be used as described by Current Protocols in Molecular Biology, (1987-2004), see e.g. Chapter 2.

### EXAMPLES

aac(6′)-Ib encodes an acetyltransferase which confer resistance to several widely used aminoglycosides antibiotics. Mutational properties of the wild-type and of a synthetic sequence derived from this gene are described below. It was established from the very start of years 1960 that nucleotidic composition of the genome of a given organism is directly reflected in its amino acid composition of its proteins (Sueoka N (1961) P.N.A.S. (USA) 47;1 141-1149). We observed that this imprint influences the evolutionary landscape which can be explored by simple change starting from a given gene, i.e., to constrain the range of amino acids accessible by simple change from a codon. We thus propose a principle of systematic handling of any gene, founded on the redundancy of the code genetic and allowing determining the sequence of genes coding for identical proteins but offering a different evolutionary landscape.

This principle allows, for example, the identification of, nucleotide sequences the most different as possible from that of the initial gene. For each codon of a given gene, one can indeed determine to it alternate codons that code for the same amino acid but which will have an altered evolutionary power, that is to say either higher, smaller or merely different. The definition of the evolutionary power depends on the constraints that one want to impose on the sequence evolutionary landscape. It can correspond to the number of amino acids accessible by simple change from a codon (“intrinsic evolutionary power”), to be defined in a more restrictive way as the number of amino acids present in the evolutionary landscape of the alternate codon which did not form part of that of the initial codon (“relative evolutionary power”) or even be calculated following a specific table set up by the designer according to his needs (“qualitative evolutionary power”). This change of coding theoretically makes possible to reach mutants which would normally require at least two changes in the same codon of the wild type gene to be able to be selected. Such double mutants of the same codon are obtained at very weak frequencies, whatever is the protocol of mutagenesis used and this even if iterative mutagenesis protocols starting from the mutants obtained are envisaged. Indeed, that would imply that the first change in the codon is at least neutral and as well as possible advantageous in term of fitness in order not to be eliminated by selection, which is absolutely not predictable. However, as this first change can be deleterious for the host, certain combinations cannot be explored by selection. One embodiment of this invention relates to a method that permits to increase specifically the number of double or triple mutations affecting some codons.

Two models have been successively developed in order to demonstrate the validity of this method. First, a synthetic gene was derived from the gene of the dehydrofolate reductase coded by gene dfrB 1, which provides resistance to the antibiotic trimethoprim. The wild-type dfrB 1 gene (further referred to as dfrB 1 WT) contains 52% G +C, however, the corresponding synthetic gene constructed dfrBIcc, contains 69% G+C. Both genes encode the same polypeptide sequence.

Experiences have been made starting from the dfrB1 WT gene having 52.7% GC and coding for a dehydrofolate reductase of 78 amino acids, conferring resistance to the trimethoprim (MIC 512 micg/ml).

A synthetic gene was then designed with a different evolutionary potential by imposing a % GC from 69+0.2, and the avoidance of E. coli rare codons, with a tolerance for rare codon (codon use less than 5% for the codons of a given amino acid) and a codon use optimized when compared to the codon use of Deinococcus radiodurans (a bacteria with a high % GC content)

The DfrB1GC gene was then assembled by hybridization of the six synthetic nucleotides hereafter:

| | | | |
|---|---|---|---|
| DfrC1 | TATGGAGCGCAGCAGCAACGAGGT | 0.2 | Phosphorylation |
| | GAGCAACCCGGTCGCCGGCAACTT | | 5′ |
| | CGTGTTCCCCAGCGACGCCACCTT | | |
| | CGGCATGGGCGACCG | | |
| DfrC2 | CGTGCGCAAGAAGAGCGGCGCCGC | 0.2 | Phosphorylation |
| | CTGGCAGGGCCAGATCGTGGGCTG | | 5′ |
| | GTACTGCACCAACCTGACCCCCGA | | |
| | GGGCTACGCCGTGGA | | |
| DfrC3 | GAGCGAGGCCCACCCCGGCAGCGT | 0.2 | Phosphorylation |
| | GCAGATCTACCCCGTGGCCGCCCT | | 5′ |
| | CGAGCGGATCAACTAA | | |
| DfrC4 | CGTCGCTGGGGAACACGAAGTTGC | 0.2 | Phosphorylation |
| | CGGCGACCGGGTTGCTCACCTCGT | | 5′ |
| | TGCTGCTGCGCTCCA | | |
| DfrC5 | TCAGGTTGGTGCAGTACCAGCCCA | 0.2 | Phosphorylation |
| | CGATCTGGCCCTGCCAGGCGGCGC | | 5′ |
| | CGCTCTTCTTGCGCACGCGGTCGC | | |
| | CCATGCCGAAGGTGG | | |
| DfrC6 | CGCGTTAGTTGATCCGCTCGAGGG | 0.2 | Phosphorylation |
| | CGGCCACGGGGTAGATCTGCACGC | | 5′ |
| | TGCCGGGGTGGGCCTCGCTCTCCA | | |
| | CGGCGTAGCCCTCGGGGG | | |

Then a ligation in a pTZ18R plasmid bearing a synthetic promoter Ptac, clonage sites NdeI-MluI for inserting the synthetic gene, previously digested by these enzymes.

The dfrB1wt gene has been cloned in the same sites and in an identical environment.

Both constructions have been inserted as a unique copy at metA locus of the E. coli chromosome by allelic exchange. This locus codes for an unrelated homoserine transsuccinylase, which is a very good locus to reach integration in E. coli chromosome, because it is quite stable.

Both bacterial strains dfrB1_{WT} and dfrB1_{GC} which were isogenic except for the dfrB1 alleles, were then submitted to continuous growth in selective medium (Mueller-Hinton+Trimethoprim at 37° C.) by serial transfer of 10⁹ cells, for 350 generations as described by Lenski and Travisano (1994).

Briefly, one milliliter of media containing 10⁹ cells issued from each culture cycle is inoculated with 63 ml of culture medium.

Maximal growth in such conditions allows six generations to be made. (2⁶⁼⁶⁴)

This high cell density in the inoculum warrants the presence of at least 10 mutated versions of the targeted gene and the conservation of the mutations. About 20 generations per day have been hen established.

This protocol allows the competitive selection of cells showing the best fitness in a given population. The populations obtained at the end of the 350 generations, in both allelic population were then submitted to competition by co-cultivation for 20 generations with either their own progenitor, the evolved population, or between evolved population (dfrB1WT+dfrB1GCevolved; dfrB1GC+dfrB1GC evolved: dfrB1WT+dfrB1WTevolved; dfrB1WTevolved et dfrB1GCevolved in mixes 1:1) as exemplified in the review of Elena and Lenski (2003). Whatever could be the co-cultivation considered, we found that the dfrB1GCevolved population took over all other populations by far (≧99.9%). Sequencing showed that the dfrB1GCevolved population was homogeneous and constituted of only a single clone carrying a mutation in the 8th codon of dfrB1GC, leading to a substitution of the valine residue into a methionine (V8M). P1 transduction of the dfrB1GC(V8M) allele in the WT strain MG1655, i.e., in an unselected genome context, and repetition of the co-cultivation experiments confirmed that the V8M mutation was uniquely and unambiguously responsible of the selective advantage.

The analysis of both cultures shows effectively unique mutation in the complete sequence gene+promoter, a change G into A of the first base of the codon 8 a to a substitution Val into Met in position 8 (GTG into ATG)

This mutation has been placed in its initial context by translation and the same results in co-culture experiences have been obtained. This last observation confirms that this mutation is effectively at the origin of the selective advantage.

To obtain this mutation from the original gene sequence, two point mutations would have been required: GTC into ATG. This example clearly illustrates the possible applications of this principle, which enables a considerable modulation of the evolutionary landscape that can be explored from a given gene coding for a functional protein.

Another model has been developed to further assess the efficiency of the principle. A synthetic gene was derived from the gene of the aminoglycoside acetyltransferase coded by aac(6′)-Ib, which typically provides resistance to the antibiotics tobramycin and amikacin. The wild-type aac(6′)-Ib gene (further referred to as aac(6′)-Ib_{WT}) contains 54% G +C. The corresponding synthetic gene constructed, aac(6′)-Ib_{SYN}, contains 51% G+C, in harmony with E. coli genome composition. Both genes encode the same polypeptide sequence. However, the two sequences share only 61% similarity at the nucleic acid level. On average, each codon of aac(6′)-Ib_{SYN} can lead to 1.6 amino acids that were not reachable by aac(6′)-Ib_{WT}.

The aac(6′)-Ib_{SYN} gene was then assembled by hybridization of the 16 synthetic nucleotides hereafter:

| | | | |
|---|---|---|---|
| No | Name | Sequence | Phosphorylation |

| | | | |
|---|---|---|---|
| 1. | AAC1t1 | AATTCATATGACGGAACACGATTT | Phosphorylation |
| | | GGCCATGTTGTAC | 5′ |
| 2. | AAC1t2 | GAATGGTTGAACAGAAGTCACATT | Phosphorylation |
| | | GTGGAATGGTGGGGGGGTGAGGAG | 5′ |
| | | GCTAGACCCACTTTGGCAGATGG | |
| 3. | AAC1t3 | TCCAAGAGCAATATCTTCCCTCGG | Phosphorylation |
| | | TGCTGGCCCAGGAAAGTGTGACGC | 5′ |
| | | CCTATATCGCTATGCTTAACGG | |
| 4. | AAC1t4 | TGAACCCATCGGTTACGCACAAAG | Phosphorylation |
| | | TTATGTGGCATTGGGTTCGGGTGA | 5′ |
| | | TGGTTGGTGGGAGGAGGAGACG | |
| 5. | AAC1t5 | GACCCCGGTGTCAGAGGTATTGAT | Phosphorylation |
| | | CAACTGCTTGCCAGGTTCGGGTGA | 5′ |
| | | TGGTTGGTGGGAGGAGGAGACG | |
| 6. | AAC1t6 | GACCCCGGTGTCAGAGGTATTGAT | Phosphorylation |
| | | CAACTGCTTGCCACCCAGAAGTGA | 5′ |
| | | CGAAAATTCAGACTGATCCCAG | |
| 7. | AAC1t7 | TCCCTCGAATCTTAGAGCCATTAG | Phosphorylation |
| | | ATGTTATGAAAAGGCCGGTTTCGA | 5′ |
| | | ACGTCAGGGGACGGTCACGACG | |
| 8. | AAC1t8 | CCCGACGGGCCCGCAGTTTATATG | Phosphorylation |
| | | GTGCAGACTAGACAAGCTTTTGAA | 5′ |
| | | AGAACTAGATCGGACGCATGAG | |
| 9. | AAC1b1 | CCCACCATTCCACAATGTGACTTC | Phosphorylation |
| | | TGTTCAACCATTCGTACAACATGG | 5′ |
| | | CCAAATCGTGTTCCGTCATATG | |
| 10. | AAC1b2 | TCCTGGGCCAGCACCGAGGGAAGA | Phosphorylation |
| | | TATTGCTCTTGGACATCTGCCAAA | 5′ |
| | | GTGGGTCTAGCCTCCTCACCCC | |
| 11. | AAC1b3 | CAATGCCACATAACTTTGTGCGTA | Phosphorylation |
| | | ACCGATGGGTTCACCGTTAAGCAT | 5′ |
| | | AGCGATATAGGGCGTCACACTT | |
| 12. | AAC1b4 | TGGCAAGCAGTTGATCAATACCTC | Phosphorylation |
| | | TGACACCGGGGTCCGTCTCCTCCT | 5′ |
| | | CCCACCAACCATCACCCGAACC | |
| 13. | AAC1b5 | TGGCAAGCAGTTGATCAATACCTC | Phosphorylation |
| | | TGACACCGGGGTCCGTCTCCTCCT | 5′ |
| | | CCCACCAACCATCACCCGAACC | |
| 14. | AAC1b6 | CTTTTCATAACATCTAATGGCTCT | Phosphorylation |
| | | AAGATTCGAGGGACTGGGATCAGT | 5′ |
| | | CTGAATTTTCGTCACTTCTGGG | |
| 15. | AAC1b7 | GTCTAGTCTGCACCATATAAACTG | Phosphorylation |
| | | CGGGCCCGTCGGGCGTCGTGACCG | 5′ |
| | | TCCCCTGACGTTCGAAACCGGC | |
| 16. | AAC1b8 | GATCCTCATGCGTCCGATCTAGTT | Phosphorylation |
| | | CTTTCAAAAGCTT | 5′ |

The assembly product was then ligated in a low copy number plasmid derived from pAM238 by partial deletion of polylinker and introduction of EcoRI cloning site. This plasmid carries a Plac promoter controlled by Lacd, upstream of the BaniHI-EcoRI cloning sites, in which the synthetic gene is inserted. This system allows a controlled gene expression, in conditions related to those of a chromosomal gene.

The aac(6′)-Ib_{WT} gene has been cloned in the same sites and in an identical environment.

Both sequences aac(6′)-Ib_{WT} and aac(6′)-Ib_{SYN} were subjected to mutagenesis using error-prone PCR (mutazyme II© kit, stratagene). The resulting alleles were cloned into the previously described plasmid and then transformed into E.coli. Two independent libraries exhibiting different mutation rates (around 1 mutation and 5 mutations per gene) were created for each sequence. Within a given library, each individuals were isogenic except for the aac(6′)-Ib alleles. Libraries were then screened in structured medium (Luria Broth+Agar+IPTG) in presence of an antibiotic gradient. The following aminoglycosides were used to create independent gradients: Tobramycine, Amikacine, Neomycin, Gentamicin, Isepamicin.

Enhanced resistance phenotypes are identified as a isolated colony at antibiotic concentration higher than the original MIC. Such colonies are purified. These aac(6′)-Ib alleles are then re-isolated, cloned and transformed in a naive genetic environment in order to eliminate false positive candidates. Once confirmed, resistance profiles on all five aminoglycosides and sequence of the corresponding alleles are determined.

TABLE 1
Mutation isolated are represented according to the antibiotic they have been selected
on and the version of the genes from which they are derived. The figures into brackets
refers to the increase in MIC compared to wild type versions. Codons implicated are
presented into parenthesis.

| | Tob | Neo | Amk | Gm | Isp |
|---|---|---|---|---|---|
| Aa_ini | Ø | Ø | Ø | L102S | Ø |
| | | | (101:CAA) | (102:TTA → TCA) | (55:TTA) |
| | | | | [x5] | |
| aac_syn | Ø | Ø | Q101L | Ø | L55Q |
| | | | (101:CAG → CTG) | (102:CTG) | (55:CTG → CAG) |
| | | | [x3] | | [x8] |

aac_ini: initial sequence;
aac_syn: synthetic sequence;
Tob: tobramycin;
Neo: neomycin;
Amk: amikacin;
Gm: gentamicin;
Isp: isepamicin;
Ø: no advantageous mutant identified

The results are represented in Table 1 above. Few mutations have been isolated, in spite of the enhanced exploration of the local sequence space by aac(6′)-Ib_{WT} and aac(6′)-Ib_{SYN}. This can be interpreted as a proof of the limited evolutionary perspectives of the protein, particularly on Tobramycin and Neomycin. On Amikacin, Gentamicin and Isepamicin, mutations that improved the level of resistance have been isolated. However, the two versions of the genes did not lead to the same set of variants. The aac(6′)-Ib_{WT} gene only led to isolation of a L102S mutation on gentamicin. This substitution have been widely described in clinical strains bearing the aac(6′)-Ib gene (ref). Indeed a simple transition from T to C allows TTA, encoding leucine in the wild type gene to reach TCA, encoding serine. This substitution has not been isolated from libraries of the synthetic gene. Indeed, in aac(6′)-Ib_{SYN} TTA has been changed to the synonymous codon CTG, because REP_{CTG/TTA}=4. The change from leucine to serine would then have required two mutations from CTG to TCG.

The other identified mutations have only been isolated from synthetic gene mutant libraries. The mutation Q101L induces a threefold increase of MIC on amikacin. This substitution is due to a transition from CAG to CTG. Such a substitution is possible from aac(6′)-Ib_{WT}: in this sequence glutamine is represented by CAA which can lead to leucine CTA. However, the codon CTA is weakly used in several γ-proteobacteria species where the gene aac(6′)-Ib is commonly found. Weakly used codons are known to reduce translation efficiency (accuracy and speed). CTA is then likely to be counter selected in nature, even if Q101L is otherwise advantageous. Indeed this mutation has only been described once, in association with the mutation L102S (ref).

The substitution L55Q has been isolated on isepamicin. It correspond to a direct CTG to CAG transversion in the aac(6′)-Ib_{SYN} gene. The leucine is encoded by TTA in aac(6′)-Ib_{WT}. Reaching a glutamine codon from TTA require TAA or CTA as intermediates. CTA is likely to be counter selected due to weak usage. TAA correspond to STOP in the genetic code. As a 185 amino-acids long protein is not likely to be functional when restricted to its first 55 amino-acids, STOP codon must be counter selected at position 55. The only way to access glutamine from TTA would then be through the sequence TTA→4→TTG→CTG→CAG, which is highly susceptible to genetic drift in large population of bacteria. The L55Q substitution has never been described so far, which might be taken as a proof of non accessibility in nature.

Two advantageous substitutions out of three would not has been isolated without inclusion of the aac(6′)-Ib_{SYN} gene into the directed evolution protocol developed. The rational design of an alternative sequence permits to broaden exploration of the sequence space, and hence to enhance directed evolution protocol efficiency.

Use of ELP Software to Select Oligonucleotide Sequences

A systematic principle of handling of any gene was proposed by the inventors, based on the redundancy of the code genetic and allowing to determine alternative sequences, coding for identical proteins but offering a potential landscape evolutionary different, even possibly most different possible from that from initial gene. Such alternative sequences give access by simple substitution to inaccessible amino acids since the native sequence. This protocol thus makes it possible to pass goatskin bottles certain constraints selective or stochastic in order to explore in a more extensive way the universe of the possible ones.

An algorithm was implemented, called Evolutionary Landscape Painter, able for any gene to determine alternative sequences of better Relative Evolutionary Potential (REP) compared to the wild version, even of better REP when one compared to the other in reference to the savage.

The Relative Evolutionary Potential of a codon X compared to a synonymous codon Y is defined like the cardinal of the whole of the acids amino accessible by a simple change from the codon X which is not accessible since Y. This program was used to build synthetic versions of the gene: aac(6′)-Ib, a bacterial gene of resistance to the aminoglycosides.

Directed Evolution of the Gene aac(6′)-Ib

A synthetic version of the gene aac(6′)-Ib was assembled. This gene codes for N-acetyl transferase pertaining to the super family of GNATs (GCN5-related N-acetyl transferase (Neuwald and Landsman, 1997). GNATs constitute a super-family of enzymes which catalyse the transfer of an acetyl group starting from the acetyl-CoA on primary amines carried by a large variety of acceptant molecules.

More precisely, AAC(6′)-Ib is an acetylase modifying some aminoglycosides (tobramycin, netilmicin, kanamycin and amikacin) but not of others (gentamicin, isepamycin). This gene has 185 codons (555 NT, G+C 54%). These characteristics make of it an ideal candidate to test the model, by widening it to obtain mutants recognizing new substrates.

Indeed, it is possible to select the mutants having an increased acetylating activity with respect to its natural substrates, but also to select mutants presenting a new acetylating spectrum. These last mutants present a much broader potential in term of industrial and search application that a simple increase in activity.

Four banks were built presenting increasing rates of changes starting from the synthetic gene. Four similar banks were established starting from the wild gene aac(6′)-Ib. These banks are screened on tobramycin, neomycin, kanamycin and amikacin, natural substrates of the enzyme, for an increase in activity. The screen is also carried out on gentamycin and isepamicin, in order to isolate variants having modified spectra of resistance.

No mutant with the increased capacities of resistance was identified on tobramycin, amikacin, kanamycin or neomycin. We conclude that the gene aac(6′)-Ib reached its evolutionary limits for the acetylating of its natural substrates. This result is supported by the results of a study carried out on the gene aac(6′)-Iaa (Salipante & Hall, Mol. Biol. Evol, 2003).

Several works mention the spontaneous appearance in clinical stocks of a variant gene, called aac(6′)-Ib′, allowing the acetylating of gentamicin instead of amikacin. By doing this the protein acquires the characteristics of an AAC of type II instead of type I.

This event is due to a single punctual mutation. It concerns a transition from T towards C which results in the replacement of a leucine by a serine into position 102. This mutant was found in all the banks of aac(6′)-Ib wild gene. On the other hand, none of the banks of synthetic gene allowed the isolation of said genotype, nor of any other genotype suggesting the existence of other variants able to resist to gentamycin.

A mutant was isolated whose capacities of resistance to isepamycin are increased (CMI×10). The mutation consists of the substitution of a leucine by a glutamine in position 55. This variant was only isolated starting from the banks resulting from synthetic gene. Such substitution is not reachable starting from initial gene.

Leucine is encoded there by codon TTA, but the glutamine corresponds to code CAA and CAG. On the other hand in synthetic gene, this leucine is represented by codon CTG. A conversion of T towards A thus carries out to obtaining a glutamine. Other mutants are in the course of characterization. The screen procedure proves being hard because it is difficult to isolate a genotype. Indeed the resistance conferred by the gene aac(6′)-Ib corresponds to a strategy of inactivation of antibiotic. Thus concentration in functional arrynoglycosides decreases locally during time around colonies allowing the less resistant phenotypes to grow in their turn. The coexistence of several genotypes within the same colony in structured medium were observed. This phenomenon prohibits the development of a screen based on the natural selection in medium not structured, weighing down as much handling necessary.

The results obtained until now consolidate this observation. The synthetic gene gave access to a variant showing increased resistance to isepamycin. This mutant was not obtained starting from wild gene. Moreover any natural or synthetic variant of the gene aac(6′)-Ib presenting this variation was not described in the data bases. On a deeper phylogenetic level, none AACs correlated with AAC(6′)Ib carries the described variation. Thus it seems that in nature, as at the laboratory, the L55Q mutation cannot emerge starting from wild gene.

In addition the mutation L102S was obtained driving to the replacement of a resistance to the amikacin by a resistance to gentamicin only starting from wild gene. That shows that the synthetic sequence in spite of the protocol of mutagenesis which is imposed to him cannot reach serine any more. The constraints weighing on this sequence are quite different from those being exerted on the initial sequence. From this point of view, it is possible to handle a gene in order to block its natural evolution towards variant which one wishes to avoid.

In conclusion, the application of the principle of widening the evolutionary landscape of a gene, shows the interest of the alternate gene synthesis for obtaining of new variant out of evolutionary possibilities starting from merely native genes.

### COMPUTER-IMPLEMENTED ASPECTS OF THE INVENTION

The invention encompasses computer-implemented selection of a synonymous nucleotide sequence containing at least one synonymous codon from among a multitude of such synonymous codons and includes the attribution to each codon of some structural parameters that when combined allow the selection of the best mutation depending on the evolutionary power required.

The following table shows aspects of the evolutionary landscape painter program.

### Evolutionary Landscape Painter

| INPUT | PROCESS | OUTPUT |
|---|---|---|
| Starting sequence | For each codon | General table: |
| | - determination of alternative codons | Initial codons; alternative |
| | - determination of corresponding | codons; evolutionary |
| | evolutionary power | powers |
| | Among alternative codons with the | |
| | best evolutionary power | |
| | - Systematic determination of | Range of G + C content |
| | codons with highest and lowest | reachable by the sequence |
| | G + C content | |
| | - Construction of a sequence with best | |
| | evolutionary power | |
| Definition of maximum forbidden codon number allowed G + C content desired and error allowed | 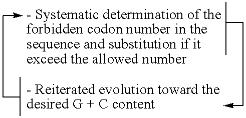 | One of the sequence with best evolutionary power which fits with imposed constraints |

The Evolutionary Landscape Painter computer program allows the determination of alternative sequences having the best relative evolutionary power (REP) for any DNA sequence written in A/T/C/G language. It is possible to select the GC content of the final sequence as well as to control the number of codons infrequently used in the final sequence.

The GC content of the genome of a particular organism is reflective of global constrains at the molecular level. It is preferable to be constrained to the GC content of the host organism in order to avoid the action of any parasitic evolutionary pressure. The computer program calculates the GC global contents of the entire sequence. Consequently, locally, the generated alternative sequences do not present a constant GC content.

Inside a genome, the use of codons is not randomly permitted. Thus, for a given amino acid, some correspondent (synonymous) codons are poorly represented. The excessive presence of such codons within a sequence could give rise to an early termination of the protein translation. Therefore, it is preferable to limit the content of such codons within the alternative sequence.

A forbidden codon is defined by the following rule. For a given amino acid, a coefficient is calculated as follows: frequency of the most used codon/frequency of the less used codon. If the value of this coefficient is higher than 6, then the codon having the slighter frequency is arbitrarily considered as having too slight a usage and is forbidden.

The ELP Program is written in PERL language. To execute it, it is necessary to have activeperl. PERL software is freely accessible at the following URL: http://www.perl.org/get.html. To use the ELP program enter the Windows command, search the file containing the ELP file and select the text file “sequence.txt”. This file corresponds to the original DNA sequence. Then type, >perl E.L.P. sequence.txt (1). The program will prompt the entry of the following data:

1. The number “N” of the forbidden codons tolerated in the final sequence. 2. The GC content “P” searched in the final sequence and 3. The threshold or error “E” tolerated for the GC content.

The output may be printed as a text file by typing: >output text” at the end of the command line (1) before executing the program.

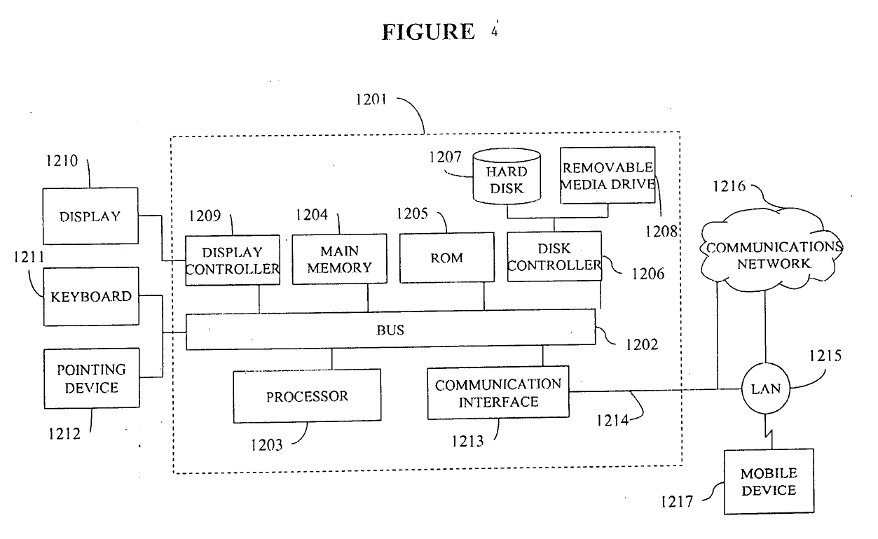

FIG. 4 illustrates a computer system 1201 upon which an embodiment of the present invention may be implemented. The computer system 1201 includes a bus 1202 or other communication mechanism for communicating information, and a processor 1203 coupled with the bus 1202 for processing the information. The computer system 1201 also includes a main memory 1204, such as a random access memory (RAM) or other dynamic storage device (e.g., dynamic RAM (DRAM), static RAM (SRAM), and synchronous DRAM (SDRAM)), coupled to the bus 1202 for storing information and instructions to be executed by processor 1203. In addition, the main memory 1204may be used for storing temporary variables or other intermediate information during the execution of instructions by the processor 1203. The computer system 1201 further includes a read only memory (ROM) 1205 or other static storage device (e.g., programmable ROM (PROM), erasable PROM (EPROM), and electrically erasable PROM (EEPROM)) coupled to the bus 1202 for storing static information and instructions for the processor 1203.

The computer system 1201 also includes a disk controller 1206 coupled to the bus 1202 to control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display 1210, such as a cathode ray tube (CRT), for displaying information to a computer user. The computer system includes input devices, such as a keyboard 1211 and a pointing device 1212, for interacting with a computer user and providing information to the processor 1203. The pointing device 1212, for example, may be a mouse, a trackball, or a pointing stick for communicating direction information and command selections to the processor 1203 and for controlling cursor movement on the display 1210. In addition, a printer may provide printed listings of data stored and/or generated by the computer system 1201.

The computer system 1201 performs a portion or all of the processing steps of the invention in response to the processor 1203 executing one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system 1201, for driving a device or devices for implementing the invention, and for enabling the computer system 1201 to interact with a human user (e.g., print production personnel). Such software may include, but is not limited to, device drivers, operating systems, development tools, and applications software. Such computer readable media further includes the computer program product of the present invention for performing all or a portion (if processing is distributed) of the processing performed in implementing the invention.

The computer code devices of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The term “computer readable medium” as used herein refers to any medium that participates in providing instructions to the processor 1203 for execution. A computer readable medium may take many forms, including but not limited to, non-volatile media, volatile media, and transmission media. Non-volatile media includes, for example, optical, magnetic disks, and magneto-optical disks, such as the hard disk 1207 or the removable media drive 1208. Volatile media includes dynamic memory, such as the main memory 1204. Transmission media includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be carried on a magnetic disk of a remote computer. The remote computer can load the instructions for implementing all or a portion of the present invention remotely into a dynamic memory and send the instructions over a telephone line using a modem. A modem local to the computer system 1201 may receive the data on the telephone line and use an infrared transmitter to convert the data to an infrared signal. An infrared detector coupled to the bus 1202 can receive the data carried in the infrared signal and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216 use, for example, electrical, electromagnetic, or optical signals that carry digital data streams, and the associated physical layer (e.g., CAT 5 cable, coaxial cable, optical fiber, etc). The signals through the various networks and the signals on the network link 1214 and through the communication interface 1213, which carry the digital data to and from the computer system 1201 maybe implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214 and the communication interface 1213. Moreover, the network link 1214 may provide a connection through a LAN 1215 to a mobile device 1217 such as a personal digital assistant (PDA) laptop computer, or cellular telephone.

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display 1210, such as a cathode ray tube (CRT), for displaying information to a computer user. The computer system includes input devices, such as a keyboard 1211 and a pointing device 1212, for interacting with a computer user and providing information to the processor 1203. The pointing device 1212, for example, may be a mouse, a trackball, or a pointing stick for communicating direction information and command selections to the processor 1203 and for controlling cursor movement on the display 1210. In addition, a printer may provide printed listings of data stored and/or generated by the computer system 1201.

The computer system 1201 performs a portion or all of the processing steps of the invention in response to the processor 1203 executing one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to the teachings of the invention and for containing data structures, tables, records, or other data described herein. Examples of computer readable media are compact discs, hard disks, floppy disks, tape, magneto-optical disks, PROMS (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes, a carrier wave (described below), or any other medium from which a computer can read.

Stored on any one or on a combination of computer readable media, the present invention includes software for controlling the computer system 1201, for driving a device or devices for implementing the invention, and for enabling the computer system 1201 to interact with a human user (e.g., print production personnel). Such software may include, but is not limited to, device drivers, operating systems, development tools, and applications software. Such computer readable media further includes the computer program product of the present invention for performing all or a portion (if processing is distributed) of the processing performed in implementing the invention.

The computer code devices of the present invention may be any interpretable or executable code mechanism, including but not limited to scripts, interpretable programs, dynamic link libraries (DLLs), Java classes, and complete executable programs. Moreover, parts of the processing of the present invention may be distributed for better performance, reliability, and/or cost.

The term “computer readable medium” as used herein refers to any medium that participates in providing instructions to the processor 1203 for execution. A computer readable medium may take many forms, including but not limited to, non-volatile media, volatile media, and transmission media. Non-volatile media includes, for example, optical, magnetic disks, and magneto-optical disks, such as the hard disk 1207 or the removable media drive 1208. Volatile media includes dynamic memory, such as the main memory 1204. Transmission media includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be carried on a magnetic disk of a remote computer. The remote computer can load the instructions for implementing all or a portion of the present invention remotely into a dynamic memory and send the instructions over a telephone line using a modem. A modem local to the computer system 1201 may receive the data on the telephone line and use an infrared transmitter to convert the data to an infrared signal. An infrared detector coupled to the bus 1202 can receive the data carried in the infrared signal and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an asymmetrical digital subscriber line (ADSL) card, an integrated services digital network (ISDN) card or a modem to provide a data communication connection to a corresponding type of communications line. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216use, for example, electrical, electromagnetic, or optical signals that carry digital data streams, and the associated physical layer (e.g., CAT 5 cable, coaxial cable, optical fiber, etc). The signals through the various networks and the signals on the network link 1214 and through the communication interface 1213, which carry the digital data to and from the computer system 1201 maybe implemented in baseband signals, or carrier wave based signals. The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214 and the communication interface 1213. Moreover, the network link 1214 may provide a connection through a LAN 1215 to a mobile device 1217 such as a personal digital assistant (PDA) laptop computer, or cellular telephone. See also, FIG. 4.

### An Example of How ELP Works

The synthesis of two alternative sequences is enough to explore all the sequences having the same evolutionary power. The first output result is random but, in selecting a second sequence, one takes in account the first generated sequence. For each amino acid, it exists at the maximum three codon having different evolutionary landscapes. If two alternative sequences are constructed with ELP there are three alternative sequences:
the original sequence
the first alternative sequence and
the second alternative sequence.

An amino acid can be imagined in a position n for which it can be found three codons with different evolutionary powers: c1, c2 and c3. Now, if the original sequence bears a codon c1, then ELP will be choose c2 or c3 randomly for the first alternative sequence and, during the determination of the second alternative sequence, ELP will take into account both, the first original sequence (bearing c1) , but also the first alternative one (bearing c2. It will not have another choice than that of selecting the third alternative codon c3. This is the reason why the synthesis of two alternative sequences is enough to explore the whole possibilities.

On the contrary, one can not to take in account the combinatory related to the incorporation of codons:

if the first original sequence bears in a position “n” an alternative codon cn1 and in position “m” an alternative codon cm1 and on the second sequence cn2 and cm2, one could imagine other alternative sequences with combinations (cn1,cm2) or (cn2, cm1) only if the amino acids placed at those position would have different evolutionary powers. It's impossible to extrapolate this to the all codons at the whole positions. The huge number of combinations would require millions of synthetic sequences.

An example of the ELP program and its program output is provided in the attached CD, whose contents are incorporated by reference.

Copyright Notice

A portion of the disclosure of this patent document contains material which is subject to copyright protection. All copyright rights whatsoever are reserved. However, the patent document may be reproduced in xerographic form in exactly the form that it appears in the Patent and Trademark Office public records.

Modifications and Other Embodiments

Various modifications and variations of the described methods as the concept of the invention will be apparent to those skilled in the art without departing from the scope and spirit of the invention. Although the invention has been described in connection with specific preferred embodiments, it should be understood that the invention as claimed is not intended to be limited to such specific embodiments. Various modifications of the described modes for carrying out the invention which are obvious to those skilled in the computer and programming arts, informatics, molecular biological, biological, chemical, medical, pharmaceutical or related fields are intended to be within the scope of the following claims.

Incorporation by Reference

Each document, patent, patent application or patent publication cited by or referred to in this disclosure is incorporated by reference in its entirety. Specifically, the disclosure of U.S. Provisional Application 60/610,597, filed Sep. 17, 2004, is hereby incorporated by reference in its entirety. However, no admission is made that any such reference constitutes prior art and the right to challenge the accuracy and pertinence of the cited documents is reserved.

1. A method for identifying a nucleotide sequence which encodes the same polypeptide as an original nucleotide sequence, but which has an altered mutational capacity, comprising:
identifying an original nucleotide sequence which encodes a polypeptide;
determining at least one synonymous nucleotide sequence encoding the same protein, which comprises at least one synonymous codon different from the corresponding codon in the original nucleotide sequence.

2. The method of claim 1, wherein at least one codon of the synonymous nucleotide sequence has a different evolutionary landscape from the corresponding codon in the original nucleotide sequence.

3. The method of claim 1, wherein at least one codon of the synonymous nucleotide sequence has a greater potential to mutate into a different amino acid by a single point mutation than the corresponding original codon.

4. The method of claim 1, wherein at least one codon of the synonymous nucleotide sequence has a lesser potential to mutate into a different amino acid by a single point mutation than the corresponding original codon.

5. The method of claim 1, further comprising synthesizing the synonymous nucleotide sequence.

6. The method of claim 1, further comprising introducing at least one point mutation into said synonymous nucleotide sequence.

7. The method of claim 6, comprising expressing the mutated synonymous nucleotide sequence and selecting a sequence encoding a polypeptide having a desired functional activity.

8. The method of claim 7, wherein said mutated synonymous nucleotide sequence is expressed in a host cell.

9. The method of claim 7, wherein a polypeptide having the functional activity of the polypeptide encoded by the original polynucleotide sequence is selected.

10. The method of claim 7, wherein a polypeptide having a lesser degree of the functional activity of the polypeptide encoded by the original polynucleotide is selected.

11. The method of claim 7, wherein a polypeptide having a greater degree of the functional activity of the polypeptide encoded by the original polynucleotide is selected.

12. The method of claim 7, wherein a polypeptide having a more stable functional activity than that of the polypeptide encoded by the original polynucleotide is selected.

13. The method of claim 1, which is a computer-implemented method.

14. The method of claim 1, which is performed using the ELP.

15. A computer-implemented method for selecting a nucleotide sequence which is synonymous to a known polynucleotide sequence, comprising:
determining the relative evolutionary potential of one or more codons in the original polynucleotide sequence, and
building at least one synonymous sequence having a higher or lower relative evolutionary potential than the known polynucleotide sequence.

16. The method of claim 15, further comprising determining at least one alternative codon having a higher or lower GC content than the original codon.

17. The method of claim 15, which comprises:
obtaining an original nucleotide sequence which encodes a polypeptide;
determining synonymous nucleotides for each codon of the sequence;
determining the intrinsic evolutionary power of each synonymous codon;
selecting a synonymous nucleotide sequence having a higher or lower intrinsic evolutionary power than the original nucleotide sequence.

18. The method of claim 15, further comprising the alternative sequences having the highest or lowest GC content.

19. A computer program for identifying a nucleotide sequence which is synonymous to a known polynucleotide sequence, comprising:
code for determining the relative evolutionary potential of one or more codons in the original polynucleotide sequence, and
code for building at least one synonymous sequence having a higher or lower relative evolutionary potential than the known polynucleotide sequence.

20. The ELP computer program.

21. A computer-readable medium comprising the computer program of claim 19.

22. A polynucleotide sequence comprising the synonymous nucleotide sequence obtained by the method of claim 1.

23. The polynucleotide of claim 22 which has been modified to have the maximum intrinsic evolutionary power.

24. The polynucleotide of claim 22, which has been modified to have the maximum relative evolutionary power.

25. The polynucleotide of claim 22, which has been modified to have the maximum intrinsic or relative evolutionary power permissible, when forbidden codons for a particular host organism in which said sequence is to be expressed are excluded from the permissible modifications.

26. The polynucleotide of claim 22, which has been modified to have the maximum intrinsic or relative evolutionary power permissible when the polynucleotide sequence is constrained to have approximately the same GC content of a particular host organism in which the polynucleotide sequence is to be expressed.

27. The polynucleotide of claim 22, in which the modifications have been determined by the ELP program.

28. A vector comprising the polynucleotide sequence of claim 22.

29. A host cell comprising the polynucleotide sequence of claim 22.

30. A polynucleotide comprising a dfBR1 polynucleotide sequence which has been modified to increase its intrinsic evolutionary power or its relative evolutionary power.

31. The polynucleotide of claim 30, which has been modified based on a synonymous polynucleotide sequence determined by the ELP program.

32. The polynucleotide of claim 30 which has been modified to have the maximum intrinsic evolutionary power.

33. The polynucleotide of claim 30, which has been modified to have the maximum relative evolutionary power.

34. The polynucleotide of claim 30, which has been modified to have the maximum intrinsic or relative evolutionary power permissible, when forbidden codons for a particular host organism in which said sequence is to be expressed are excluded from the permissible modifications.

35. The polynucleotide of claim 30, which has been modified to have the maximum intrinsic or relative evolutionary power permissible when the polynucleotide sequence is constrained to have approximately the same GC content of a particular host organism in which the polynucleotide sequence is to be expressed.

36. A vector comprising the polynucleotide sequence of claim 30.

37. A host cell comprising the vector of claim 36.

38. A process for preparing a mutated nucleic acid comprising mutated codons encoding the identical amino acid sequence that the wild type or original nucleic acid encodes which comprises:
identifying a nucleic acid sequence synonymous with that of the wild-type or original nucleic acid sequence by the method of claim 1, and
synthesizing the synonymous nucleic acid sequence.

39. A method for making a mutant polypeptide comprising,
determining a synonymous polynucleotide for a native, wild-type or original polypeptide encoding polynucleotide according to the method of claim 1,
synthesizing said synonymous polynucleotide sequence,
transforming said synonymous polynucleotide sequence into a host cell,
culturing said host cell under conditions in which point mutations may accumulate in said synonymous polynucleotide sequence and optionally under conditions favorable for selection of mutant cells containing mutations in said synonymous polynucleotide sequence,
isolating a mutant cell expressing a mutant polypeptide, and
recovering said mutant polypeptide.

40. A polypeptide obtained by the method of claim 39, which is optionally encoded by:
a polynucleotide sequence having at least 90% similarity to that of the synonymous polynucleotide sequence or the original polynucleotide sequence from which the synonymous sequence was derived, or
which hybridizes under stringent conditions to the synonymous or original polynucleotide sequence encoding the original, unmodified polypeptide.


SEQUENCE LISTING (22 sequences)

SEQ ID NO: 1 - DNA, 37 bp, artificial sequence
  AAC1t1 chemically synthesized
aattcatatgacggaacacgatttggccatgttgtac

SEQ ID NO: 2 - DNA, 70 bp, artificial sequence
  AAC1t2 nucleotide chemically synthesized
gaatggttgaacagaagtcacattgtggaatggtgggggggtgaggaggctagacccactttggcagatg

SEQ ID NO: 3 - DNA, 70 bp, artificial sequence
  AAC1t3 nucleotide chemically synthesized
tccaagagcaatatcttccctcggtgctggcccaggaaagtgtgacgccctatatcgctatgcttaacgg

SEQ ID NO: 4 - DNA, 70 bp, artificial sequence
  AAC1t4 nucleotide chemically synthesized
tgaacccatcggttacgcacaaagttatgtggcattgggttcgggtgatggttggtgggaggaggagacg

SEQ ID NO: 5 - DNA, 70 bp, artificial sequence
  AAC1t5 nucleotide chemically synthesized
gaccccggtgtcagaggtattgatcaactgcttgccaggttcgggtgatggttggtgggaggaggagacg

SEQ ID NO: 6 - DNA, 70 bp, artificial sequence
  AAC1t6 nucleotide chemically synthesized
gaccccggtgtcagaggtattgatcaactgcttgccacccagaagtgacgaaaattcagactgatcccag

SEQ ID NO: 7 - DNA, 70 bp, artificial sequence
  AAC1t7 nucleotide chemically synthesized
tccctcgaatcttagagccattagatgttatgaaaaggccggtttcgaacgtcaggggacggtcacgacg

SEQ ID NO: 8 - DNA, 70 bp, artificial sequence
  AAC1t8 nucleotide chemically synthesized
cccgacgggcccgcagtttatatggtgcagactagacaagcttttgaaagaactagatcggacgcatgag

SEQ ID NO: 9 - DNA, 70 bp, artificial sequence
  AAC1b1 nucleotide chemically synthesized
cccaccattccacaatgtgacttctgttcaaccattcgtacaacatggccaaatcgtgttccgtcatatg

SEQ ID NO: 10 - DNA, 70 bp, artificial sequence
  AAC1b2 nucleotide chemically synthesized
tcctgggccagcaccgagggaagatattgctcttggacatctgccaaagtgggtctagcctcctcacccc

SEQ ID NO: 11 - DNA, 70 bp, artificial sequence
  AAC1b3 nucleotide chemically synthesized
caatgccacataactttgtgcgtaaccgatgggttcaccgttaagcatagcgatatagggcgtcacactt

SEQ ID NO: 12 - DNA, 70 bp, artificial sequence
  AAC1b4 nucleotide chemically synthesized
tggcaagcagttgatcaatacctctgacaccggggtccgtctcctcctcccaccaaccatcacccgaacc

SEQ ID NO: 13 - DNA, 70 bp, artificial sequence; identical to SEQ ID NO: 12
  AAC1b5 nucleotide chemically synthesized

SEQ ID NO: 14 - DNA, 70 bp, artificial sequence
  AAC1b6 nucleotide chemically synthesized
cttttcataacatctaatggctctaagattcgagggactgggatcagtctgaattttcgtcacttctggg

SEQ ID NO: 15 - DNA, 70 bp, artificial sequence
  AAC1b7 nucleotide chemically synthesized
gtctagtctgcaccatataaactgcgggcccgtcgggcgtcgtgaccgtcccctgacgttcgaaaccggc

SEQ ID NO: 16 - DNA, 37 bp, artificial sequence
  AAC1b8 nucleotide chemically synthesized
gatcctcatgcgtccgatctagttctttcaaaagctt

SEQ ID NO: 17 - DNA, 87 bp, artificial sequence
  DfrC1 nucleotide chemically synthesized
tatggagcgcagcagcaacgaggtgagcaacccggtcgccggcaacttcgtgttccccagcgacgccaccttcggcatgggcgaccg

SEQ ID NO: 18 - DNA, 87 bp, artificial sequence
  DfrC2 nucleotide chemically synthesized
cgtgcgcaagaagagcggcgccgcctggcagggccagatcgtgggctggtactgcaccaacctgacccccgagggctacgccgtgga

SEQ ID NO: 19 - DNA, 64 bp, artificial sequence
  DfrC3 nucleotide chemically synthesized
gagcgaggcccaccccggcagcgtgcagatctaccccgtggccgccctcgagcggatcaactaa

SEQ ID NO: 20 - DNA, 63 bp, artificial sequence
  DfrC4 nucleotide chemically synthesized
cgtcgctggggaacacgaagttgccggcgaccgggttgctcacctcgttgctgctgcgctcca

SEQ ID NO: 21 - DNA, 87 bp, artificial sequence
  DfrC5 nucleotide chemically synthesized
tcaggttggtgcagtaccagcccacgatctggccctgccaggcggcgccgctcttcttgcgcacgcggtcgcccatgccgaaggtgg

SEQ ID NO: 22 - DNA, 90 bp, artificial sequence
  DfrC6 nucleotide chemically synthesized
cgcgttagttgatccgctcgagggcggccacggggtagatctgcacgctgccggggtgggcctcgctctccacggcgtagccctcggggg